(19) United States
(12) Patent Application Publication
(10) Pub. No.: US 2014/0081373 A1
(43) Pub. Date: Mar. 20, 2014

(54) STENT WITH RETENTION PROTRUSIONS FORMED DURING CRIMPING

(71) Applicant: Abbott Cardiovascular Systems Inc., Santa Clara, CA (US)

(72) Inventors: Timothy A. Limon, Cupertino, CA (US); Bin Huang, Pleasanton, CA (US); Vincent J. Gueriguian, San Francisco, CA (US)

(73) Assignee: Abbott Cardiovascular Systems Inc., Santa Clara, CA (US)

(21) Appl. No.: 14/082,062

(22) Filed: Nov. 15, 2013

Related U.S. Application Data
(63) Continuation of application No. 11/445,736, filed on Jun. 1, 2006.

(51) Int. Cl. A61F 2/915; A61F 2/958

(52) U.S. Cl. CPC A61F 2/915; A61F 2/958
USPC 623/1.11; 623/1.16; 29/515

(57) ABSTRACT

Stents that forms protrusions in a crimped state and methods of crimping the stent are disclosed.

### CROSS-REFERENCE TO RELATED APPLICATION

This application is a continuation of U.S. patent application Ser. No. 11/445,736, filed Jun. 1, 2006, the entire disclosure of which is incorporated herein by reference.

### BACKGROUND OF THE INVENTION

1. Field of the Invention

This invention relates to polymeric stents and methods of delivery of polymeric stents.

2. Description of the State of the Art

This invention relates to radially expandable endoprostheses, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty. “Restenosis” refers to the reoccurrence of stenosis in a blood vessel or heart valve after it has been subjected to angioplasty or valvuloplasty.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity. Once expanded, the stent must adequately maintain its size and shape throughout its service life despite the various forces that may come to bear on it, including the cyclic loading induced by the beating heart.

A stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed to allow crimping and radially expanded to allow deployment, which will be described below.

Additionally, it may be desirable for a stent to be biodegradable. In many treatment applications, the presence of a stent in a body may be necessary for a limited period of time until its intended function of, for example, maintaining vascular patency and/or drug delivery is accomplished. Thus, stents are often fabricated from biodegradable, bioabsorbable, and/or bioerodable materials such that they completely erode only after the clinical need for them has ended.

In the case of a balloon expandable stent, the stent is mounted about a balloon disposed on a catheter. Mounting the stent typically involves compressing or crimping the stent onto the balloon. The stent must be retained on the balloon during delivery until it is deployed at an implant or treatment site within a vessel in the body of a patient. The stent is then expanded by inflating the balloon. “Delivery” refers to introducing and transporting the crimped stent through a bodily lumen to the treatment site in a vessel. “Deployment” corresponds to the expanding of the crimped stent within the lumen at the treatment site. Delivery and deployment of a stent are accomplished by positioning the stent about one end of a catheter, inserting the end of the catheter through the skin into a bodily lumen, advancing the catheter in the bodily lumen to a desired treatment location, inflating the stent at the treatment location, and removing the catheter from the lumen by deflating the balloon.

The crimped stent on the balloon-catheter assembly must have a small delivery diameter so that it can be transported through the narrow passages of blood vessels. The stent must also be firmly attached to the catheter to avoid detachment of the stent before it is delivered and deployed in the lumen of the patient. Detachment of a stent from the catheter during delivery and deployment can result in medical complications. A lost stent can act as an embolus that can create a thrombosis and require surgical intervention. For this reason, a stent must be securely attached to the catheter.

Stent retention is greatly facilitated by protrusion or penetration of the balloon into the interstitial spaces or gaps between stent struts in a stent pattern when the stent is crimped onto the balloon. However, for polymeric stents the degree of penetration, and thus stent retention, in polymeric stents can be lower than metallic stents due to larger strut size in polymeric stents. In order to have adequate mechanical strength, polymeric stents may require significantly thicker struts than a metallic stent. The wider struts provide less space for a balloon to protrude through when the stent is crimped onto a delivery balloon.

### SUMMARY

Certain aspects of the present invention include embodiments of a stent including a plurality of interconnecting structural elements, the structural elements including a bending element configured to bend to allow crimping of the stent, the bending element having an angle between about 110° to 150°, wherein a protrusion forms on a luminal surface of the bending element when the stent is crimped.

Further aspects of the invention include a stent including a plurality of interconnecting structural elements, the structural elements including a bending element configured to bend to allow crimping of the stent, wherein a protrusion forms on a luminal surface of the bending element when the stent is crimped, wherein a thickness of the protrusion normal to the luminal surface is at least 10% of a thickness of the bending element when the stent is in an uncrimped state.

Additional aspects of the invention include a method of crimping a stent including providing a stent including a plurality of interconnecting structural elements, the structural elements including a bending element configured to bend to allow crimping of the stent, the bending element having an angle between about 110° to 150°, wherein protrusions form on an abluminal side and a luminal surface of the bending element when the stent is crimped; disposing the stent over a balloon positioned on a catheter; crimping the stent over the balloon so that the angle of the bending element is between about 0° and 30′; and allowing protrusions to form during crimping on a luminal side of the bending element, wherein the protrusions contact the balloon in such a way to facilitate retention of the stent on the balloon during delivery of the stent into a bodily lumen.

### BRIEF DESCRIPTION OF THE DRAWINGS

FIG. 1 depicts a stent.

FIG. 2 depicts a view of a bending element from the stent of FIG. 1 in an uncrimped state.

FIG. 3 depicts an exemplary embodiment of a stent of the present invention

FIG. 4 depicts a view of a bending element from the stent of FIG. 3 in an uncrimped state.

FIG. 5 depicts a view of a bending element from the stent of FIG. 4 in a crimped state.

FIG. 6A depicts a balloon in a deflated state disposed over a catheter.

FIG. 6B depicts a radial cross-section of a crimped stent over a balloon.

FIG. 6C depicts a close-up view of an apex region of a bending element of a crimped stent.

FIG. 7 depicts a bending element.

FIGS. 8-9 are photographs of a crimped stent of the present invention.

### DETAILED DESCRIPTION OF THE INVENTION

Those of ordinary skill in the art will realize that the following description of the invention is illustrative only and not in any way limiting. Other embodiments of the invention will readily suggest themselves to such skilled persons based on the disclosure herein. All such embodiments are within the scope of this invention.

For the purposes of the present invention, the following terms and definitions apply:

As used herein, the term “radius of curvature” refers to the length of a line segment extending from the center of a circle or sphere to the circumference or bounding surface, or the circular area defined by a stated radius.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Modulus” may be defined as the ratio of a component of stress or force per unit area applied to a material divided by the strain along an axis of applied force that results from the applied force. For example, a material has both a tensile and a compressive modulus. A material with a relatively high modulus tends to be stiff or rigid. Conversely, a material with a relatively low modulus tends to be flexible. The modulus of a material depends on the molecular composition and structure, temperature of the material, amount of deformation, and the strain rate or rate of deformation. For example, below its Tg, a polymer tends to be brittle with a high modulus. As the temperature of a polymer is increased from below to above its Tg, its modulus decreases.

A polymer for use in fabricating an implantable medical device, such as a stent, can be biostable, bioabsorbable, biodegradable or bioerodable. Biostable refers to polymers that are not biodegradable. The terms biodegradable, bioabsorbable, and bioerodable are used interchangeably and refer to polymers that are capable of being completely degraded and/or eroded when exposed to bodily fluids such as blood and can be gradually resorbed, absorbed and/or eliminated by the body. The processes of breaking down and absorption of the polymer can be caused by, for example, hydrolysis and metabolic processes.

It is understood that after the process of degradation, erosion, absorption, and/or resorption has been completed, no part of the stent will remain or in the case of coating applications on a biostable scaffolding, no polymer will remain on the device. In some embodiments, very negligible traces or residue may be left behind. For stents made from a biodegradable polymer, the stent is intended to remain in the body for a duration of time until its intended function of, for example, maintaining vascular patency and/or drug delivery is accomplished.

Representative examples of polymers that may be used to fabricate an implantable medical device include, but are not limited to, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(L-lactide-co-glycolide); poly(D,L-lactide), poly(caprolactone), poly(trimethylene carbonate), polyethylene amide, polyethylene acrylate, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose.

Additional representative examples of polymers that may be especially well suited for use in fabricating an implantable medical device according to the methods disclosed herein include ethylene vinyl alcohol copolymer (commonly known by the generic name EVOH or by the trade name EVAL), poly(butyl methacrylate), poly(vinylidene fluoride-co-hexafluoropropene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol.

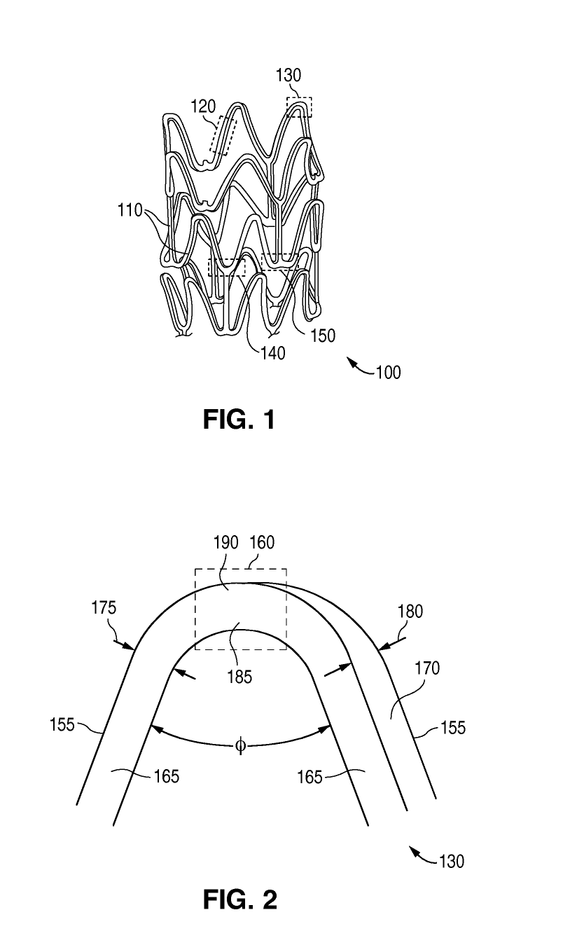

A stent can include a pattern of a plurality of interconnecting structural elements or struts. FIG. 1 depicts an example of a view of a stent 100. Stent 100 includes a pattern with a number of interconnecting structural elements or struts 110. In general, a stent pattern is designed so that the stent can be radially compressed (crimped) and radially expanded (to allow deployment). The stresses involved during compression and expansion are generally distributed throughout various structural elements of the stent pattern.

As shown in FIG. 1, the geometry or shape of stent 100 varies throughout its structure to allow radial expansion and compression. A pattern may include portions of struts that are straight or relatively straight, an example being a portion 120. In addition, patterns may include struts that include bending elements as in sections 130, 140, and 150. Bending elements bend inward when a stent is crimped to allow radial compression. Bending elements also bend outward when a stent is expanded to allow for radial expansion.

In some embodiments, a stent may be fabricated by laser cutting a pattern on a tube. Representative examples of lasers that may be used include, but are not limited to, excimer, carbon dioxide, and YAG. In other embodiments, chemical etching may be used to form a pattern on a tube. An outside diameter (OD) of a stent or a polymer tube prior to fabrication of a stent is typically between about 1 mm and about 3 mm. Thus, the OD of a fabricated or uncrimped stent can be between about 0.04 in and about 0.12 in. When a stent is crimped, the structural elements deform allowing the stent to decrease in diameter. The deformation occurs primarily at bending elements which bend inward. One method of crimping involves disposing a stent over a balloon that is disposed over a support member such as a catheter. The balloon may be partially inflated to allow the stent to conform to the balloon. Inward radial pressure is applied to the stent by devices known in the art to compress the stent over the balloon.

Various embodiments of the invention include a stent having protrusions that form on at least the luminal surface of the bending elements of a stent due to compression as the stent is crimped. In particular, the protrusions form in the apex regions of the bending elements. The embodiments also include methods of crimping a stent that form such protrusions. Such protrusions facilitate stent retention on a balloon. The protrusions on the luminal surface of a stent press against the balloon when the stent is crimped over the balloon, improving retention of the stent on the balloon during delivery of the stent to a bodily lumen.

FIG. 2 depicts a view of a bending element 130 from stent 100 in an uncrimped state that includes straight sections 155 and a curved or apex section 160 with an angle φ. Bending element 130 has a luminal surface 165, an abluminal surface (not shown), and a sidewall surface 170. Bending element 130 can have a width 175 and a thickness 180. When a stent is crimped, angle φ decreases and concave portion 185 experiences relatively high compressive strain and convex portion 190 experiences relatively high tensile strain. Due to the compression in concave portion 185, stent material can protrude outward from the abluminal and luminal surfaces of the concave portion. In general, the greater the change in bending angle causes more compression which increases the size of the protrusions.

Thus, the size of protrusions depends in part upon the change in bending angle of bending elements from the uncrimped state to the crimped state and the diameter of the stent in the uncrimped state. The diameter of the stent in the uncrimped state must be large enough to allow for a selected change in angle of the bending element. For example, if the diameter is too small, the stent will reach the crimped diameter before the bending element reaches the selected change in angle. Typically, a balloon mounted on a catheter has an outside diameter of between about 0.028 in (0.737 mm) and 0.032 in (0.813 mm). An outside diameter of a crimped stent is approximately the outside diameter of the balloon.

Certain embodiments of the invention include stents having bending elements with angles between 80° to 150°, 100° to 150°, or more narrowly, between 120° to 150°. The stent may have an uncrimped diameter that allows the stent to be crimped to a selected crimped diameter at which the bending elements have an angle between 0° to 50°, or more narrowly between 0° to 50°. In some embodiments, the crimped diameter may be less than 0.04 in, 0.036 in, 0.032 in, or more narrowly less than 0.028 in. In some embodiments, the OD of an uncrimped stent may be between 0.07 in and 0.165 in. In other embodiments the OD of an uncrimped stent may be greater than 0.165 in.

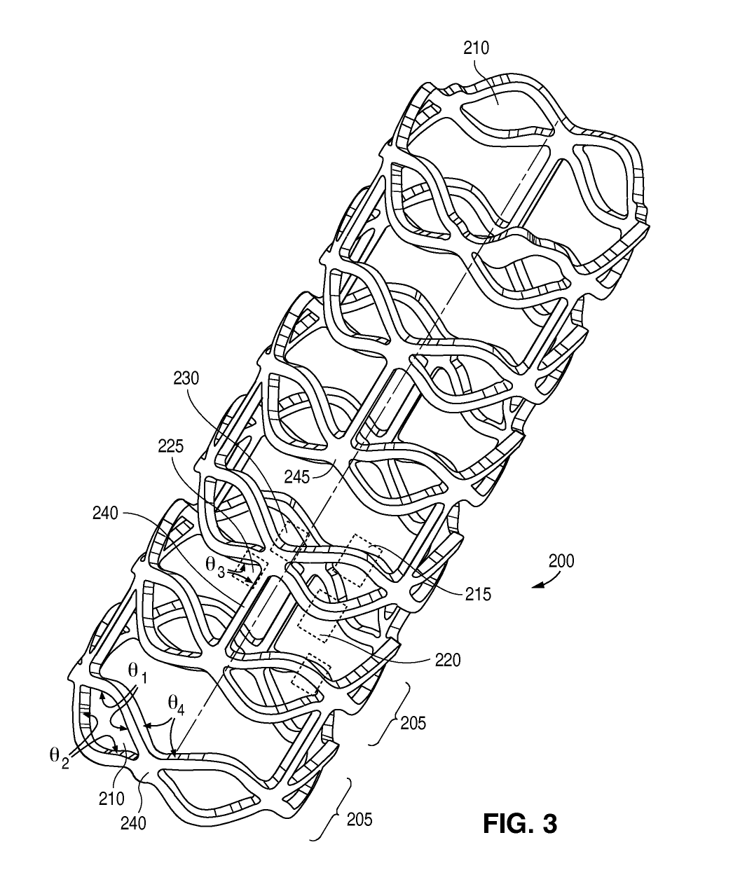

FIG. 3 depicts an exemplary embodiment of a stent 200 of the present invention. As depicted in FIG. 3, stent 200 includes a plurality of cylindrical rings 205 with each ring including a plurality of diamond shaped cells 210. Diamond shaped cells 210 include bending elements 215 and 220. Stent 200 can also include bending elements 225 and 230. The angles of bending elements 215, 220, 225, and 230 correspond to θ₁, θ₂, θ₃, and θ₄.

Pattern 200 further includes linking arms 240 that connect adjacent cylindrical rings. Linking arms 240 are parallel to the longitudinal axis of the stent and connect adjacent rings between intersections 245 of cylindrically adjacent diamond-shaped elements 210.

When stent 200 is crimped, bending elements 215, 220, 225, and 230 flex inward and angles θ₁, θ₂, θ₃, and θ₄ decrease, allowing the stent to be radially compressed. With respect to bending elements 215, 220, and 230, struts on either side of the bending elements bend toward each other. However, in bending element 225, the strut of the diamond-shaped element tends to bend toward the linking strut which tends to remain relatively parallel to the longitudinal axis during crimping.

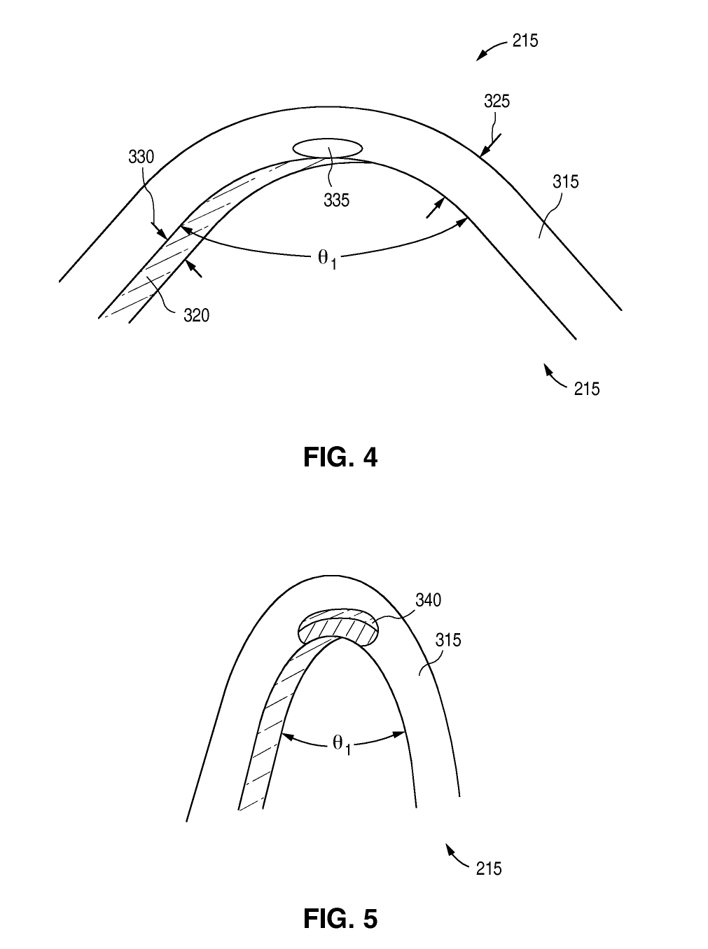

FIG. 4 depicts a view of bending element 215 of stent 200 in an uncrimped state. Bending element 210 has a luminal surface 315, an abluminal surface (not shown), and a sidewall surface 320. Bending element 215 can have a width 325 and a thickness 330. Width 325 may be between about 0.012 in and 0.02 in, or more narrowly between 0.002 in and 0.007 in.

FIG. 5 depicts a view of bending element 215 in a crimped state. In the crimped state, angle θ₁ decreases and concave portion 335 from FIG. 4 experiences relatively high compressive strain which causes a protrusion 340 on luminal surface 315 at concave portion 335. Protrusions also form at the luminal surfaces of bending elements 220, 225, and 230. In some embodiments, the thickness of the protrusion normal to luminal surface 315 can be greater than 5%, 10%, or 15% of thickness 330 of the bending element 215 in an uncrimped state.

Bending elements 215 and 220 have angles between about 80° to 150°, 100° to 150°, or more narrowly, between 120° to 150° in an uncrimped state. Also, bending elements 215 and 220 can have radii of curvature between 0.010 in and 0.025 in. In the crimped state, bending elements 215 and 220 have angles between 0° to 30° and radii of curvature between 0.0005 in and 0.005 in. The OD of an uncrimped stent can be between 0.07 in and 0.165 in and the crimped diameter can be between 0.032 in and 0.055 in.

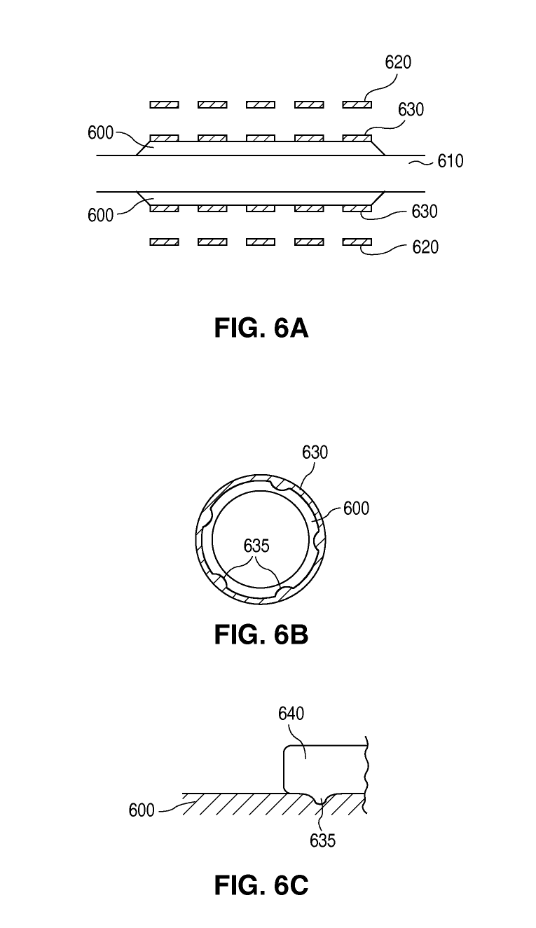

As indicated above, the protrusions tend to facilitate retention of a crimped stent on a balloon. FIG. 6A depicts an axial cross-section of a balloon 600 in a deflated state disposed over a catheter 610. An uncrimped stent 620 is disposed over balloon 600. Stent 620 is crimped over the outside surface of balloon 600, as shown by crimped stent 630, by methods known to those of skill in the art. Typically, an inward radial pressure is applied to uncrimped stent 620 to cause a decrease in diameter. FIG. 6B depicts a radial cross-section of crimped stent 630 over balloon 600. Protrusions 635 protrude into balloon 600. FIG. 6C shows a close-up view of an apex region 640 of a bending element of crimped stent 630. Apex region 640 shows protrusion 635 protruding into the surface of balloon 600.

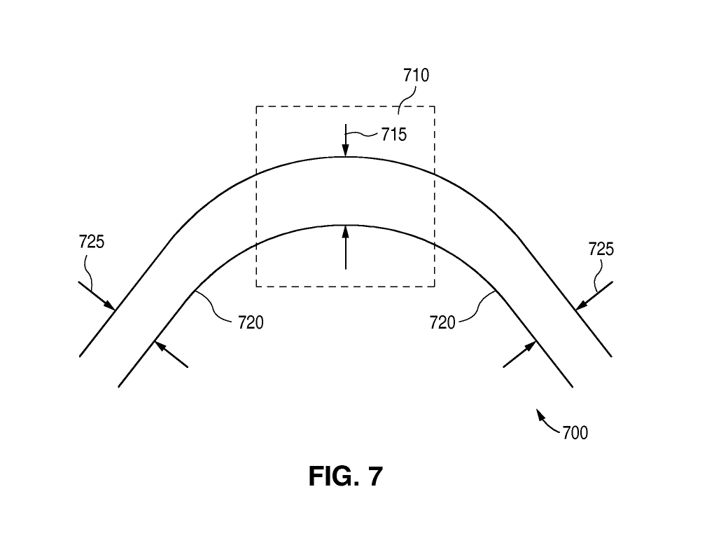

In some embodiments, the thickness or size of the protrusion can be increased by selectively increasing the mass of the apex region of a bending element. For example, the width at an apex region can be larger than other regions of the stent pattern. FIG. 7 depicts a bending element 700 having an apex region 710 with a thickness 715. Thickness 715 is greater than thickness 725 of section 720 of bending element 700. The increased mass in the apex regions results in compression of more material during crimping which increases the size of a protrusion. The increased size of the protrusions further enhances stent retention on a balloon.

Additionally, polymers having a higher tensile modulus than compressive modulus tend to result in larger protrusions. Furthermore, the size of the protrusions can be further increased by using polymers having a tensile modulus substantially higher than a compressive modulus. For example, a tensile modulus substantially higher than compressive modulus may refer to a tensile modulus 30%, 50%, 100%, or 200% higher than a compressive modulus.

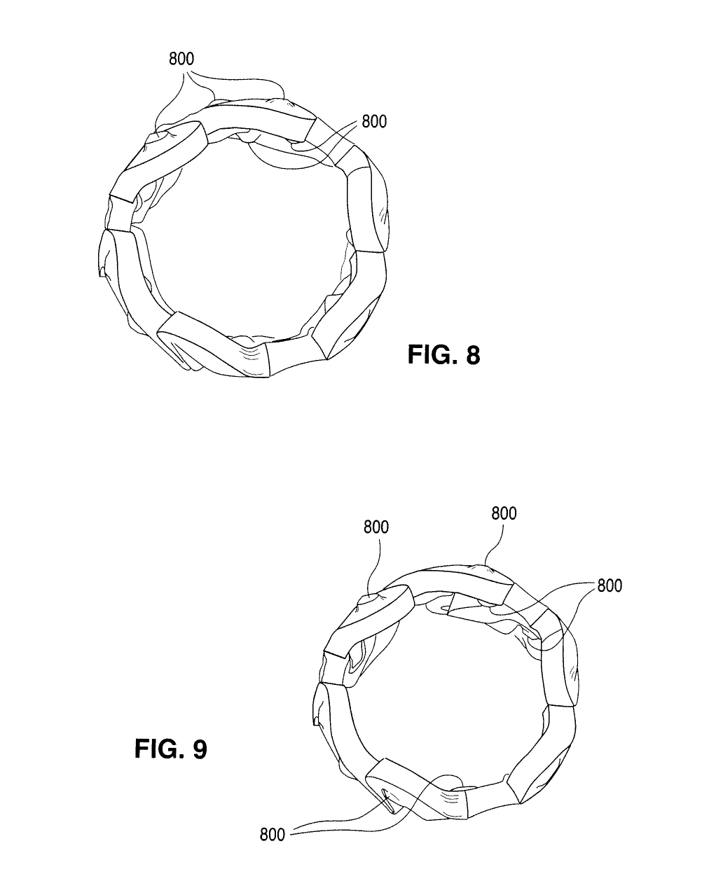

FIGS. 8-9 are photographs of a crimped stent of the present invention with views down the longitudinal axis of the stent. As shown in both FIGS. 9 and 10, the stent has protrusions 800 on the luminal and abluminal surface of bending elements.

While particular embodiments of the present invention have been shown and described, it will be obvious to those skilled in the art that changes and modifications can be made without departing from this invention in its broader aspects.

1-18. (canceled)

19. An endoprosthesis comprising:
circumferential rings of struts and longitudinal linking struts connecting the rings, wherein the rings form a cylindrical scaffold having a first end and a second end, wherein the scaffold comprises a polymer including poly(L-lactide),
wherein the rings include a first ring, a second ring adjacent the first ring, and a third ring adjacent the first ring, the first ring disposed between the second ring and the third ring, each of the rings having a plurality of bending elements that bend inward when the scaffold is crimped and bend outward when the scaffold is expanded, each of the bending elements having apex sections,
wherein in each of the rings, the apex sections alternate between pointing toward the first end of the scaffold and pointing toward the second end of the scaffold,
wherein each apex section of the first ring is located opposite a corresponding apex section of the second ring, both of which apex sections are longitudinally aligned and directed toward the same end of the scaffold,
wherein each apex section of the first ring is located opposite a corresponding apex section of the third ring, both of which apex sections are longitudinally aligned and directed toward the same end of the scaffold,
wherein each apex section of the first ring is connected by a linking strut to a corresponding apex section of the second ring or the third ring, and wherein the linking struts alternate between connecting the first ring to the second ring and connecting the first ring to the third ring,
wherein the scaffold has a fabricated state and a crimped state, wherein the scaffold is radially compressed from the fabricated state to the crimped state for delivery of the scaffold into a vessel,
wherein the bending elements have an angle between 80° and 150° when the scaffold is in the fabricated state.

20. The endoprosthesis of claim 19, wherein the angle is between 100° and 150° when the scaffold is in the fabricated state.

21. The endoprosthesis of claim 19, wherein the angle is between 120° and 150° when the scaffold is in the fabricated state.

22. The endoprosthesis of claim 19, wherein the scaffold has an outer diameter of between 0.07 inch and 0.165 inch in the fabricated state.

23. The endoprosthesis of claim 19, wherein the scaffold has an outer diameter of between 0.04 inch and 0.12 inch in the fabricated state.

24. The endoprosthesis of claim 19, wherein the scaffold has an outer diameter greater than 0.165 inch in the fabricated state.

25. The endoprosthesis of claim 19, wherein the polymer comprises poly(L-lactide-co-glycolide).

26. An method of making an endoprosthesis comprising:
providing a scaffold comprising circumferential rings of struts and longitudinal linking struts connecting the rings, wherein the rings form a cylindrical scaffold having a first end and a second end, wherein the scaffold comprises a polymer including poly(L-lactide),
wherein the rings include a first ring, a second ring adjacent the first ring, and a third ring adjacent the first ring, the first ring disposed between the second ring and the third ring, each of the rings having a plurality of bending elements, each of the bending elements having apex sections,
wherein in each of the rings, the apex sections alternate between pointing toward the first end of the scaffold and pointing toward the second end of the scaffold,
wherein each apex section of the first ring is located opposite a corresponding apex section of the second ring, both of which apex sections are longitudinally aligned and directed toward the same end of the scaffold,
wherein each apex section of the first ring is located opposite a corresponding apex section of the third ring, both of which apex sections are longitudinally aligned and directed toward the same end of the scaffold,
wherein each apex section of the first ring is connected by a linking strut to a corresponding apex section of the second ring or the third ring, and wherein the linking struts alternate between connecting the first ring to the second ring and connecting the first ring to the third ring,
wherein the apex sections of the bending elements have an angle between 80° and 150° when the scaffold is in a fabricated state; and
crimping the scaffold from the fabricated state to a crimped state onto a balloon,
wherein each of the bending elements bends inward when the scaffold is crimped to allow radial compression of the scaffold that decreases a diameter of the scaffold to the crimped state,
wherein the bending elements have an angle between 0° and 50° when the scaffold is in the crimped state.

27. The method of claim 26, wherein the angle in the fabricated state is between 100° and 150° when the scaffold is in the fabricated state.

28. The method of claim 26, wherein the angle in the fabricated state is between 120° and 150° when the scaffold is in the fabricated state.

29. The method of claim 26, wherein the scaffold has an outer diameter of between 0.07 inch and 0.165 inch in the fabricated state.

30. The method of claim 26, wherein the scaffold has an outer diameter of between 0.04 inch and 0.12 inch in the fabricated state.

31. The method of claim 26, wherein the scaffold has an outer diameter greater than 0.165 inch in the fabricated state.

32. The method of claim 26, wherein the polymer comprises poly(L-lactide-co-glycolide).

33. A method of treating stenosis in a blood vessel comprising:
providing a scaffold comprising a polymer including poly(L-lactide) mounted over a balloon in a crimped state that is crimped from a fabricated state, wherein the scaffold includes circumferential rings of struts and longitudinal linking struts connecting the rings, wherein the rings form a cylindrical scaffold having a first end and a second end,
wherein the rings include a first ring, a second ring adjacent the first ring, and a third ring adjacent the first ring, the first ring disposed between the second ring and the third ring, each of the rings having a plurality of bending elements, each of the bending elements having apex sections,
wherein in each of the rings, the apex sections alternate between pointing toward the first end of the scaffold and pointing toward the second end of the scaffold,
wherein each apex section of the first ring is located opposite a corresponding apex section of the second ring, both of which apex sections are longitudinally aligned and directed toward the same end of the scaffold,
wherein each apex section of the first ring is located opposite a corresponding apex section of the third ring, both of which apex sections are longitudinally aligned and directed toward the same end of the scaffold,
wherein each apex section of the first ring is connected by a linking strut to a corresponding apex section of the second ring or the third ring, and wherein the linking struts alternate between connecting the first ring to the second ring and connecting the first ring to the third ring,
wherein the bending elements have an angle between 80° and 150° when the scaffold is in a fabricated state and an angle between 0° and 50° when the scaffold is in the crimped state,
introducing the mounted scaffold into a blood vessel;
transporting the mounted scaffold to a site of stenosis in the blood vessel; and
expanding and implanting the mounted scaffold at the site of stenosis by inflating the balloon which expands the site of stenosis in the blood vessel,
wherein the bending elements bend outward when the scaffold is expanded to allow radial expansion of the scaffold, and
wherein the implanted scaffold maintains patency of the blood vessel at the site for a period of time.

34. The method of claim 33, wherein the implanted scaffold completely erodes away after the clinical need to maintain patency is completed.

35. The method of claim 33, wherein the angle in the fabricated state is between 100° and 150° when the scaffold is in the fabricated state.

36. The method of claim 33, wherein the angle in the fabricated state is between 120° and 150° when the scaffold is in the fabricated state.

37. The method of claim 33, wherein the scaffold has an outer diameter of between 0.07 inch and 0.165 inch in the fabricated state.

38. The method of claim 33, wherein the scaffold has an outer diameter greater than 0.165 inch in the fabricated state.

39. The method of claim 33, wherein the polymer comprises poly(L-lactide-co-glycolide).
